(19) United States
(12) Patent Application Publication
(10) Pub. No.: US 2010/0129924 A1
(43) Pub. Date: May 27, 2010

(54) Quality assurance method for olefin polymerization catalysts

(76) Inventor: Sandor Nagy, Naperville, IL (US)

Correspondence Address: LyondellBasell Industries, 3801 WEST CHESTER PIKE, NEWTOWN SQUARE, PA, 19073, US

(21) Appl. No.: 12/313,575

(22) Filed: Nov. 21, 2008

(51) Int. Cl. G01N 33/00

(52) U.S. Cl. 436/141

(57) ABSTRACT

A method for evaluating the quality of an olefin polymerization catalyst is disclosed. A single-site organometallic complex is reacted with a methyl alumoxane under controlled reaction conditions. The extent and/or dynamics of methane formation from the reaction are measured, and the results are used to predict the suitability of the complex and/or the methyl alumoxane for use in an olefin polymerization.

### FIELD OF THE INVENTION

The invention relates to single-site catalysts used to make polyolefins. In particular, the method relates to a way to ensure consistent production of high-quality catalysts.

### BACKGROUND OF THE INVENTION

Single-site catalysts, which include metallocenes and non-metallocenes, are a diverse class of systems used to polymerize olefins. Most of the known catalyst systems include an organometallic complex and an activator. Common activators include alumoxanes, alkyl aluminum compounds, and ionic borates. When alumoxanes activate the catalyst, the principal active site for polymerization is believed to be a cationic transition metal center—stabilized by an alumoxane anion—that complexes olefins and fosters chain growth.

Side reactions complicate the seemingly simple process, however. For instance, when metallocenes are activated by alumoxanes, methane can form (see Eisch et al., Eur. J. Org. Chem. (2005) 4364). Numerous mechanisms for methane generation have been proposed (see, e.g., Kaminsky et al., J. Polym. Sci. A 42 (2004) 3911; Polyhedron 7 (1988) 2375; and J. Mol. Catal. 128 (1998) 191). Based on nuclear magnetic resonance (NMR) spectroscopy studies, others have suggested that methane might result from decomposition of a bimetallic zirconium species (see, e.g., J. Organometal. Chem. 484 (1994) C10; Organometallics 22 (2003) 33; Organometallics 20 (2001) 2088; and J. Am. Chem. Soc. 126 (2004) 1448). Our own molecular modeling results (reported herein) support the idea of a bridged heterobimetallic adduct as an intermediate that forms during methane generation.

Reasonable people will disagree about why and how methane forms, but most will agree that the catalyst decomposition, evidenced in part by methane generation, undermines performance. This may manifest itself in poor catalyst activity, broad polymer molecular weight distribution (because of multiple and different active sites), or an increase in polymer long-chain branching.

Despite the bounty of literature related to single-site olefin polymerization catalysts, particularly ones activated using methyl alumoxanes, relatively little attention has been devoted to quality assurance. Most of the related art focuses on combinatorial or high-throughput experimentation techniques and polymer characterization (see, e.g., U.S. Pat. No. 6,998,269). Additionally, Kappler et al. (Polymer 44 (2003) 6179) reported a high-throughput technique to monitor ethylene/1-hexene copolymerization in which FT-IR is used to monitor catalyst activity, polymerization kinetics, 1-hexene conversion, and other parameters. Thus, earlier approaches rely on costly olefin polymerization experiments to predict catalyst performance or to monitor quality. Moreover, these techniques are generally limited to small-scale preparations of complexes and are not well-suited to real-time monitoring of a catalyst scale-up operation.

The polyolefin industry would benefit from convenient, inexpensive ways to monitor the quality of a single-site polymerization catalyst. Preferably, the likely performance of the catalyst could be evaluated without the need to perform polymerization experiments. Ideally, the method could be conducted “on line” during even a full-scale production of a single-site catalyst to ensure the manufacture of a consistent, high-quality product.

### SUMMARY OF THE INVENTION

The invention relates to a method for evaluating the quality of an olefin polymerization catalyst. The method comprises three steps. First, a single-site organometallic complex is reacted with a methyl alumoxane under controlled reaction conditions. Second, the extent and/or dynamics of methane formation from the reaction are measured. Finally, the results of this measurement are used to predict the suitability of the complex and/or the methyl alumoxane for use in an olefin polymerization.

### DETAILED DESCRIPTION OF THE INVENTION

The quality of an olefin polymerization catalyst is evaluated by measuring the tendency an organometallic complex to form methane when the complex is combined with a methyl alumoxane under controlled conditions. The measurement provides information useful for predicting the suitability of the complex and/or the methyl alumoxane for use in an olefin polymerization. The inventive method comprises three steps, which are outlined below.

First, a single-site organometallic complex is reacted with a methyl alumoxane under controlled reaction conditions. By “controlled reaction conditions,” we mean that important reaction parameters are monitored and maintained within acceptable limits so that meaningful comparisons can be made. These parameters might include, for example, temperature, pressure, order of addition of reactants, addition times, reaction time, complex and methyl alumoxane concentration, amount and kind of any support used, pretreatment routine used for the support, and other factors.

Suitable single-site organometallic complexes are those that can be used with methyl alumoxane activators to give catalysts useful for polymerizing olefins. Suitable complexes incorporate a Group 3-10 transition metal. Preferably, the complexes incorporate an early transition metal, i.e., a Group 4 to 6 transition metal, more preferably a Group 4 metal. Suitable complexes include, for example, metallocenes, constrained-geometry complexes (e.g., monocyclopentadienyl silylamide complexes), heterometallocenes, indenoindolyl complexes, complexes containing chelating ligands (diamides, salicylamidinates, benzamidinates), late transition metal complexes (bisimines), and any other complex that can react with a methyl alumoxane to form a catalytically active species. For examples of suitable single-site organometallic complexes, see U.S. Pat. Nos. 6,232,260; 6,451,724; 6,693,154; 5,324,800; 6,670,297; 5,637,660; 6,204,216; 6,498,221; and 5,064,802, the teachings of which related to the complexes and their methods of preparation are incorporated herein by reference.

The complexes react with a methyl alumoxane. Suitable methyl alumoxanes are well known, and many are commercially available. Usually, methyl alumoxanes are supplied as a 4-30 wt. % mixture of the alumoxane in an aliphatic or aromatic hydrocarbon solvent. Examples include MAO (methyl-aluminoxane, product of Albemarle), which is available as a 10 wt. % or 30 wt. % solution in toluene, and PMAO (polymethylaluminoxane, solution in toluene), a product of Akzo Nobel. Modified methyl alumoxanes, such as MMAO-3A, MMAO-7, and MMAO-12 (products of Akzo Nobel), and similar materials are also suitable. Suitable modified methyl alumoxanes incorporate 5-30 mole % of a C₂-C₈ alkyl group and 70-95 mole % methyl groups. Methylalumoxane (MAO or PMAO) is preferred.

The amount of methyl alumoxane used relative to the amount of complex varies and depends on many factors, including the nature of the complex and the methyl alumoxane, whether the complex is supported or not, solvent effects, the particular method being used to measure the rate or amount of methane generated, and other factors. Generally, the amount used is enough to generate a measurable amount of methane. Usually, the amount will be similar to that used in practice for an olefin polymerization process. Thus, the amount used will generally be within the range of about 1 to about 5000 moles, preferably from about 10 to about 500 moles, and more preferably from about 50 to about 200 moles, of aluminum per mole of transition metal.

In the first method step, the single-site organometallic complex and the methyl alumoxane are reacted under controlled conditions. In a second step, the extent and/or dynamics of methane formation from the reaction of the complex and the methyl alumoxane are measured. Any desired technique or combination of techniques can be used to measure methane formation. As those skilled in the art will appreciate, any of a variety of analytical techniques, alone or in combination, might be exploited to evaluate methane formation from the reaction of a single-site organometallic complex and a methyl alumoxane. Examples include gas chromatography (GC), Raman spectroscopy, Fourier transform infrared spectroscopy (FTIR), proton or carbon-13 nuclear magnetic resonance spectroscopy (¹H or ¹³C NMR), mass spectrometry, or combinations of these. Gas chromatograpy is preferred.

Third, the results of the measurement of methane formation are used to predict the suitability of the single-site organometallic complex and/or the methyl alumoxane for use in an olefin polymerization. Suitability relates to one or more parameters considered important for evaluating a single-site catalyst and/or activator and their likely performance characteristics. For example, the suitability might relate to catalyst activity, i.e., how a particular complex will behave as an olefin polymerization catalyst when it is combined with a particular activator. A complex that generates a relatively high concentration of methane when combined with a methyl alumoxane should have a relatively low activity, which may or may not be desirable.

Suitability of a particular complex might relate to how easily the resulting polyolefin can be processed, which in turn relates to the molecular weight distribution of the polyolefin and/or the amount of long-chain branching present in the polyolefin. Because elevated methane levels indicate that a catalyst is developing multiple active sites, one can infer that complexes that generate a relatively high concentration of methane will also provide polymers having broadened molecular weight distributions and/or higher levels of long-chain branching. Thus, if polymers with enhanced processability are desired, it might be desirable to select catalysts that generate at least a modest amount of methane when they are combined with a methyl alumoxane.

The following examples illustrate the invention. The skilled person will, of course recognize other particular ways to design experiments to effectively evaluate catalyst quality using methane monitoring methods of the invention.

### EXAMPLE 1

### Complex Screening

A standard set of parameters is selected and used to evaluate a series of different single-site organometallic complexes. Each complex is tested by combining the same molar amount of complex with the same molar amount of the same methyl alumoxane under the same conditions of temperature, pressure, reaction solvent, reaction time, and so on, to appreciate differences in the tendency of these complexes to form methane under identical conditions. If desired, a combinatorial or high-throughput technique can be used to screen a large number of complexes and to select the best one for further evaluation.

### EXAMPLE 2

### Batch-to-Batch Consistency

A standard apparatus is used repeatedly to prepare and test multiple batches of a particular supported single-site organometallic complex. Each batch is prepared using an identical procedure. Measurement of the tendency of each batch of complex to form methane provides information helpful for quality control, such as the amount of batch-to-batch variation to expect from a particular catalyst preparation recipe.

### EXAMPLE 3

### Finding the Right Alumoxane

A series of methyl alumoxanes from different sources is reacted with a single organometallic complex under a standard set of reaction conditions to understand the tendency of a particular methyl alumoxane to form methane. This technique allows the skilled person to select a methyl alumoxane that best fits the organometallic complex.

### EXAMPLE 4

### Aging Effects

Aging effects are identified by activating, under a standard set of conditions with a single methyl alumoxane, samples from a catalyst preparation batch that have aged for different amounts of time. These results help to inform a decision about the storage stability of a complex and how soon it will need to be used following its preparation.

### EXAMPLE 5

### Activator to Complex Ratio

Methane formation is used to help select a desirable ratio of activator to complex. A particular organometallic complex and a particular methyl alumoxane are reacted under a standard set of reaction conditions, except that the molar ratio of complex to methyl alumoxane is varied, and methane formation is measured.

### EXAMPLE 6

### Reactor and Process Design Improvements

Samples are drawn simultaneously from different locations within a commercial reactor during the reaction of the complex and the methyl alumoxane to identify areas of local overheating (i.e., “hot spots”) in the reactor. The results inform decisions about possible need for better mixing, better moisture exclusion, addition of baffles, or an improved reactor design.

### EXAMPLE 7

### Kinetic Studies

Samples are collected from the headspace of a reactor during catalyst activation, preferably as a function of time, then analyzed by gas chromatography to quantify the amount of methane present in each sample. Kinetics of the process are determined and compared with similar information from experiments with other complexes, other batches of the same complex, other methyl alumoxanes, or other investigations. The results reveal the dynamics of methane formation from the reaction of the complex and the methyl alumoxane.

### EXAMPLE 8

### Storage Stability

Samples are collected from the headspace of sealed containers of catalyst and analyzed by gas chromatography to determine the amount of methane present in the headspace. By tracking and recording how catalysts perform as a function of the amount of methane measured in the headspace of stored samples, one can predict whether a particular catalyst sample will be acceptable for use in an olefin polymerization process.

### EXAMPLE 9

### Moisture Content

Methane generation is used to determine whether the moisture content of a complex, a supported catalyst, or their combination with a particular reaction solvent is too high. Because water reacts with methyl alumoxanes to generate methane, a relatively high level of methane could indicate that better moisture control is needed for one or more of these components.

### EXAMPLE 10

### Evaluation of Supports

For most olefin polymerizations, complexes are supported on silicas, aluminas, or other inorganic oxides. Often, the support is treated with a methyl alumoxane before it is combined with a complex. Methane is produced when the alumoxane reacts with surface hydroxyl groups of the support. Methane generation can be measured for a series of different supports to understand which support is most desirable.

### EXAMPLE 11

### Support Pre-Treatment Methods

Silicas and other supports are often calcined or chemically modified prior to use in supporting a complex. The effectiveness of these pre-treatment methods can be evaluated using methane monitoring. For example, addition of a methyl alumoxane under standard conditions to silicas calcined for a set time period at several different temperatures can reveal how effective each calcination temperature was for reducing the surface hydroxyl group content of the silica.

### EXAMPLE 12

### Alumoxane-Treated Support

Alumoxanes are commonly used to pre-treat a support prior to combining the support with an organometallic complex. The amount of methyl alumoxane needed to provide a treated support that maximizes catalyst activity can be determined using methane monitoring.

### EXAMPLE 13

### Incipient Wetness Preparation

In the incipient wetness technique, a supported complex is prepared by adding a minimum amount of: (1) a solution of complex to a thermally or chemically treated support; or (2) a solution of complex and methyl alumoxane to a thermally or chemically treated support. Each method generates a supported catalyst as a dry powder. Methane generation measured in the process of making catalysts by incipient wetness can be correlated with catalyst activity and used for quality control.

### EXAMPLE 14

### Alumoxane and Complex

Generation of methane is normally undesirable when a complex (or a supported complex) and a methyl alumoxane are combined. Thus, methane monitoring can identify whether better process control or moisture exclusion is needed at this stage of the catalyst preparation process.

### EXAMPLE 15

### Alumoxane-Treated Support and Complex

Generation of methane is normally undesirable when a methyl alumoxane-treated support and a complex are combined. Thus, methane monitoring can identify whether better process control or moisture exclusion is needed at this stage of the catalyst preparation process.

Methane Generation: A Modeling Study

Consider the simple case of methylalumoxane (MAO) activation of bis(cyclopentadienyl)zirconium dichloride, which is believed to proceed through a zirconium dimethyl species.

Our calculations indicate that unassisted decomposition of bis(cyclo-pentadienyl)zirconium dimethyl to give methane and a zirconium carbene (Model Reaction 1) is energetically unfavorable (see Table 1):

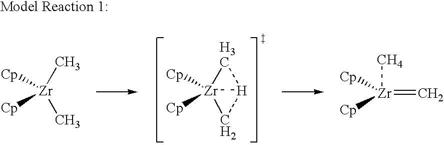

The energetics become relatively favorable when various aluminum-containing species (likely to form when MAO reacts with the complex) can stabilize the resulting zirconium carbene by forming a heterobimetallic adduct, as shown in Model Reactions 2-5 (see also Table 1):

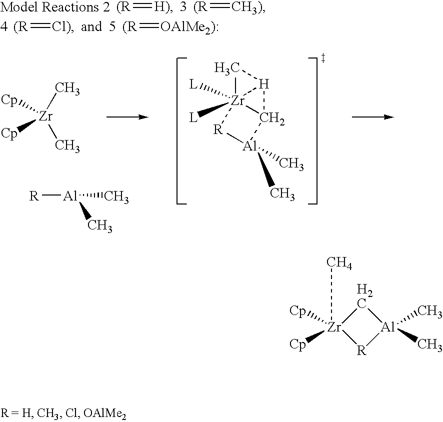

Methane might be generated in an alternative unassisted process involving bimetallic deactivation of two transition metal centers (Model Reaction 6), but this is also energetically unfavorable (see Table 1):

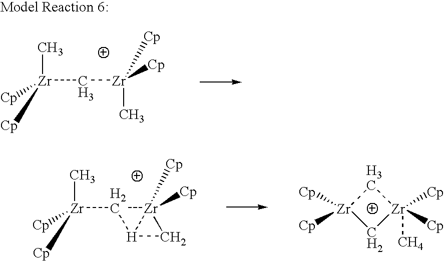

Calculations are performed using the B3LYP DFT method using a 6-31G** (pseudopotential) as implemented in the Spartan '06 software of Wavefunction, Inc. The potential energy surfaces are explored by constraining two distances (“reaction coordinates”) in a series of calculations to generate a grid in the vicinity of the transition state. Results appear in Table 1.

TABLE 1
Calculated Energies of Model Reactions

| | | Energy Relative to Ground | | |
|---|---|---|---|---|
| | | State (kcal/mol) | | Barrier of |
| | | H-Transfer | Product | Reverse |
| | Model | Transition State | Energy | Reaction |
| | Reaction | Energy (kcal/mol) | (kcal/mol) | (kcal/mol) |
| Cp₂ZrMe₂ | 1 | 44 | 28 | 16 |
| Cp₂ZrMe₂/Me-AlMe₂ | 2 | 31 | −10 | 41 |
| Cp₂ZrMe₂/H—AlMe₂ | 3 | 25 | −8 | 33 |
| Cp₂ZrMe₂/Cl—AlMe₂ | 4 | 21 | −22 | 43 |
| Cp₂ZrMe₂/(Me₂AlO)—AlMe₂ | 5 | 24 | −21 | 45 |
| Cp₂ZrMe--Me--ZrMeCp₂ | 6 | 36 | −3 | 39 |

As shown in Table 1, the transition state energies for hydrogen transfer are more favorable when a methylene-bridged heterobimetallic (Zr—Al) species stabilized by a second bridging fragment (CH₃, H, Cl, or OAlR₂) can form (Model Reactions 2-5). The energetics are also favorable for limiting the reverse reaction, which has a relatively high barrier for Model Reactions 2-5.

The modeling study provides evidence that the methane generation observed experimentally may involve formation of heterobimetallic adducts.

The above examples are intended only as illustrations; the following claims define the invention.

1. A method comprising:
(a) reacting a single-site organometallic complex with a methyl alumoxane under controlled reaction conditions;
(b) measuring the extent and/or dynamics of methane formation from the reaction; and
(c) using the results of the measurement to predict the suitability of the complex and/or the methyl alumoxane for use in an olefin polymerization.

2. The method of claim 1 wherein the measurement is performed using gas chromatography, ¹H NMR spectroscopy, ¹³NMR spectroscopy, FTIR spectroscopy, Raman spectroscopy, mass spectrometry, or combinations thereof.

3. The method of claim 1 wherein the complex comprises a Group 4-6 transition metal.

4. The method of claim 1 wherein the complex is selected from the group consisting of metallocenes, constrained-geometry complexes, heterometallocenes, indenoindolyl complexes, complexes containing chelating ligands, and late transition metal complexes.

5. The method of claim 1 wherein the suitability relates to the activity of a catalyst comprising the methyl alumoxane and the complex.

6. The method of claim 1 wherein the suitability relates to the molecular weight distribution of a polyolefin made using a catalyst comprising the methyl alumoxane and the complex.

7. The method of claim 1 wherein the suitability relates to the amount or kind of long-chain branching in a polyolefin made using a catalyst comprising the methyl alumoxane and the complex.

8. The method of claim 1 wherein the methyl alumoxane is selected from the group consisting of MAO, modified MAOs, and mixtures thereof.
